(19) United States
(12) Patent Application Publication
(10) Pub. No.: US 2025/0170034 A1
(43) Pub. Date: May 29, 2025

(54) COMPOSITION FOR TOOTH WHITENING OR IMPROVING DENTIN HYPERSENSITIVITY COMPRISING POLYPHOSPHATE

(71) Applicant: LG H & H CO., LTD., Seoul (KR)

(72) Inventors: Kyo Tae MOON, Seoul (KR); Jeongyun NAM, Seoul (KR); Kyoung Hee OH, Seoul (KR)

(73) Assignee: LG H & H CO., LTD., Seoul (KR)

(21) Appl. No.: 18/934,727

(22) Filed: Nov. 1, 2024

(30) Foreign Application Priority Data
Nov. 2, 2023 (KR) 10-2023-0150208
Oct. 30, 2024 (KR) 10-2024-0150746

(51) Int. Cl. A61K 8/24; A61K 8/19; A61K 8/27; A61Q 11/00

(52) U.S. Cl. CPC A61K 8/24; A61K 8/19; A61K 8/27; A61Q 11/00

(57) ABSTRACT

The present invention relates to an oral composition comprising a potassium ion, a phosphate ion, an organic zinc compound, and a polyphosphate. The composition according to the present invention demonstrates excellent tooth whitening, tooth enamel strengthening, and improving dentin hypersensitivity, and thus, it can be effectively used for tooth whitening, tooth enamel strengthening, or improving, preventing, or treating dentin hypersensitivity.

### TECHNICAL FIELD

The present invention relates to a composition for tooth whitening or improving dentin hypersensitivity, comprising a polyphosphate, and more specifically, to a composition having a significant effect of improving dentin hypersensitivity as well as an excellent whitening effect.

### BACKGROUND ART

Bright, white smiles significantly help build good interpersonal relationships. Further, a confident smile leaves a more positive first impression.

Recently, there has been a growing interest in tooth whitening beyond oral health, and in line with this trend, toothpaste compositions with tooth whitening effects are being released.

Tooth whitening refers to a dental treatment that whitens and brightens teeth discolored due to various reasons without damaging the teeth. Tooth whitening products for home use have recently been developed.

There are various types and methods of tooth whitening, but most methods employ high concentrations of hydrogen peroxide to oxidize the staining agents on the teeth to bleach the teeth. These methods have been used in dental practices for over 100 years, and their whitening effect has been proven. Peroxides act as oxidizing agents that oxidize the teeth-staining substances, removing them through modification or degradation and thereby achieving the effect of tooth whitening; however, their storage has been limited due to high reactivity. Further, oxidation reactions using hydrogen peroxide are very irritating and cause strong irritation upon contact with the lips, gums, tongue, and the like.

Dentin hypersensitivity refers to a symptom in which the teeth become sensitive to external stimuli when the alveolar bone dissolves due to an inflammatory response caused by factors such as aging or disease, leading to a reduction in the height of the alveolar bone, gum recession, and the subsequent exposure of the dentin that should lie beneath the gums, or when the teeth become temporarily weakened or thinned due to the use of whitening toothpastes comprising hydrogen peroxide or dental whitening procedures. Methods for alleviating dentin hypersensitivity include a method of changing the oral balance of sodium ions and potassium ions, which are essential in the nerve transmission system.

As a method of changing the oral balance of the ions, a method of increasing the oral potassium ion concentration by introducing an excess of potassium ions is used, and the most commonly used substances include potassium nitrate, potassium chloride, potassium oxalate, and potassium phosphate.

Meanwhile, tooth whitening treatments or whitening toothpastes that use hydrogen peroxide are designed to have a low pH to enhance the stability and efficacy of hydrogen peroxide, and this leads to the dissolution of hydroxyapatite on the tooth surface by acid, thereby causing demineralization and weakening of the teeth. Methods of tooth strengthening include a method of remineralization using fluorine contained in the toothpaste. The fluorine strengthens the teeth by remineralizing hydroxyapatite on the tooth surface into fluorapatite through an ion exchange reaction between the hydroxy groups and the fluoride ions. However, despite undergoing such a remineralization process, there still remains an issue that weakened teeth cannot ultimately be sufficiently strengthened.

Accordingly, research is being actively conducted on pyrophosphates or polyphosphates, both of which are excellent ingredients for removing teeth-staining substances and inhibiting and preventing restaining, in place of hydrogen peroxide. However, low molecular weight polyphosphates may form chelates with dissolved calcium on the tooth surface or form chelates with supersaturated calcium in the saliva, which can cause demineralization of the teeth. Therefore, this method also cannot resolve the problem of weakening tooth strength. Meanwhile, pyrophosphates can also reduce tooth hardness by removing calcium on the tooth surface.

Further, even in the case of simply adding remineralization agents such as calcium ions or phosphate ions, which are components of the teeth, to address the issue of weakening tooth strength, there still remains the issue of reduced stability of hydrogen peroxide or a reduced whitening effect due to chelate formation by ion activity.

Under the circumstances, the present inventors conducted extensive research to address such issues. As a result, the present inventors have developed a toothpaste composition with an excellent whitening effect by comprising a potassium ion, a zinc compound, and a polyphosphate. Further, the present inventors have confirmed that the composition exhibits the effects of strengthening tooth enamel and improving dentin hypersensitivity, which can resolve consumer concerns about weakening of tooth strength or dentin hypersensitivity that may arise from tooth whitening, thereby completing the present invention.

### DISCLOSURE

### Technical Problem

An object of the present invention is to provide an oral composition comprising a potassium ion source, a phosphate ion source, an organic zinc compound, and a polyphosphate.

Another object of the present invention is to provide a pharmaceutical composition for preventing or treating dentin hypersensitivity comprising a potassium ion source, a phosphate ion source, an organic zinc compound, and a polyphosphate.

Still another object of the present invention is to provide the use of the composition comprising a potassium ion source, a phosphate ion source, an organic zinc compound, and a polyphosphate in tooth whitening, tooth enamel strengthening, or improving dentin hypersensitivity.

### Technical Solution

The present invention will be described in detail as follows. Meanwhile, each description and embodiment disclosed herein can be applied to other descriptions and embodiments, respectively. That is, all combinations of various elements disclosed herein fall within the scope of the present disclosure. Further, the scope of the present invention is not limited by the specific description described below.

The present invention relates to a composition for tooth whitening, tooth enamel strengthening, or improving dentin hypersensitivity. A composition comprising potassium ions, which can improve dentin hypersensitivity, an organic zinc compound, which can fill the gaps in weakened teeth, and a polyphosphate having whitening effect was invented. It was specifically confirmed that the composition of the present invention maintains excellent whitening effect while exhibiting tooth enamel strengthening and improvement in dentin hypersensitivity.

Hydroxyapatite, a tooth component, is formed by a reaction between calcium ions and phosphate ions contained in the saliva and/or toothpaste. Phosphate ions can play a role in strengthening the teeth by reacting with saliva and penetrating the tooth surface. Potassium phosphate and zinc stearate may help increase the hardness of the tooth surface. Zinc ions can play an important role in remineralization of tooth surface by replacing calcium and forming zinc phosphate, thereby hardening the tooth surface. Further, zinc, which has antimicrobial properties, is also known to prevent bacterial contamination and enamel loss on the tooth surface (FIG. 1).

Referring to FIG. 1, polyphosphates strongly adhere electrostatically to a tooth surface and form a film with an excess negative charge, and by introducing divalent cations that have two ion-binding ability to the formed film and thereby forming electrostatic bonds between the cations and the phosphate ions (PO₄³⁻), polyphosphates can play a role in strengthening the teeth.

The present invention relates to a composition aimed at simultaneously achieving tooth whitening and tooth enamel strengthening, which may include potassium ions that easily penetrate the micro cracks between the teeth (FIG. 2). Meanwhile, toothpastes containing potassium chloride, potassium oxalate, etc., to provide potassium ions do not have any whitening effect. While they may relieve oral irritation by increasing the concentration of potassium ions in the micro cracks between the teeth and inhibiting external stimuli such as warmth and cold from reaching the nerve transmission system of the dentin, they cannot contribute to strengthening tooth enamel.

As shown in the remineralization mechanism below, phosphate ion sources increase the supply of phosphate ions (PO₄³⁻) using Le Chatelier's principle and enhance the production of hydroxyapatite, thereby helping to fill the gaps between the teeth. However, in the tooth remineralization mechanism by phosphate ions, polyphosphates compete with calcium ions in the formation of chelates. Accordingly, phosphate ions may inhibit the whitening effect by inhibiting the attachment of polyphosphates to the teeth. Accordingly, there is a need to develop a method capable of maintaining the effects of tooth whitening and strengthening tooth enamel while containing phosphate ions.

Ca₁₀(PO₄)₆(OH)₂↔10Ca²⁺+6PO₄³⁻+2OH⁻

Additionally, when a polyphosphate and a zinc salt are included, the negatively charged portion of the polyphosphate binds to the zinc cation and forms a chelate, thereby reducing the charge density of the polyphosphate anion. This weakens the strength of the bond formed with the calcium ions on the tooth surface and may reduce the whitening effect obtained by pushing and eliminating contaminants of the teeth. Accordingly, it is very important to keep the zinc salts stable in the formulation. For example, zinc chloride, which can readily ionize in water, reacts with water to dissociate into zinc ions and chloride ions. The zinc ions form chelates with polyphosphates and thereby weaken the binding strength of polyphosphates to the tooth surface. On the other hand, organic zinc compounds, such as zinc citrate, zinc stearate, and zinc gluconate, refer to compounds in which organic acids and zinc ions are bound. When in contact with water, these compounds do not readily dissociate but remain stable through electron stabilization of the carboxyl groups in the organic acid. Accordingly, the present invention uses specifically the organic zinc compounds among zinc salts to keep the zinc salts stable in the formulation.

In the present invention, potassium ions and phosphate ions are provided either separately or simultaneously using potassium phosphate and coexist with organic zinc compounds. Thus, partially-dissociated zinc ions in the composition react with phosphate ions rather than polyphosphates, thereby maintaining the whitening effect of the polyphosphates, and strengthening tooth enamel by being delivered to the teeth after being bound in the form of zinc phosphate. Accordingly, it was confirmed that the composition of the present invention can achieve all of the effects of tooth whitening, tooth enamel strengthening, and improving dentin hypersensitivity, thereby completing the present invention.

An aspect of the present invention provides an oral composition comprising a potassium ion source, a phosphate ion source, an organic zinc compound, and a polyphosphate.

In the present invention, the composition may be used for one or more purposes selected from the group consisting of tooth whitening, tooth enamel strengthening, and improving dentin hypersensitivity.

As used herein, the term “tooth whitening” may refer to the removal of substances that attach to or penetrate the tooth surface and enamel thereby causing teeth-staining, or the removal of the staining by modifying the chemical structure of the substances.

As used herein, the term “dentin hypersensitivity” may refer to an independent disease in which transient or persistent sharp pain occurs in response to an external stimulus, independent of cavities or other pathological causes. Dentin hypersensitivity is a symptom in which an exposed part of the dentin of the teeth becomes sensitive when exposed to cold air or when in contact with irritating foods. It may occur in the following cases: dental erosion near the gums due to improper brushing habits, excessive occlusal forces, or dissolution by acids; poor oral hygiene; or dissolution of teeth due to acidification after periodontal or restorative treatment. The fundamental cause of dentin hypersensitivity is the exposure of numerous tubules present in the dentin of the teeth, which transmits all stimuli directly to the nerves within the pulp and thereby results in more sensitive reactions to stimuli than usual, potentially causing pain. Dentin hypersensitivity can range from mild symptoms to intense and persistent pain. Further, since teeth do not regenerate by nature, pain relievers, anti-inflammatory drugs, etc. do not provide a fundamental solution to dentin hypersensitivity. Dentin hypersensitivity may occur throughout the teeth or may be limited to specific areas, such as the maxilla or mandible, or the right or left side. The most commonly affected areas vary depending on the cause, but they are mainly the canines and premolars. In over 90% of the cases, the area in which the pain is most severe is known to be the cervical area, which is the junction between the gums and the teeth.

As used herein, the term “improvement” refers to the reduction or relief of dentin hypersensitivity as described above and may be understood as a broader concept, including alleviation, prevention, treatment, and the like.

In the present invention, the potassium ion source may be one or more selected from the group consisting of monopotassium phosphate (KH₂PO₄), dipotassium phosphate (K₂HPO₄), tripotassium phosphate (K₃PO₄), potassium nitrate (KNO₃), potassium chloride (KCl), and potassium oxalate (K₂C₂O₄), but is not limited thereto.

As used herein, the term “potassium ion source” may refer to a compound capable of providing potassium ions (K⁺) in the composition. For example, monopotassium phosphate (KH₂PO₄) may be referred to as the potassium ion source or the phosphate ion source because it is capable of providing both the potassium ions and the phosphate ions in the composition.

In the present invention, the phosphate ion source may be one or more selected from the group consisting of monopotassium phosphate (KH₂PO₄), dipotassium phosphate (K₂HPO₄), tripotassium phosphate (K₃PO₄), monosodium phosphate (NaH₂PO₄), disodium phosphate (Na₂HPO₄), and trisodium phosphate (Na₃PO₄), but is not limited thereto.

As used herein, the term “phosphate ion source” may refer to a compound capable of providing phosphate ions (PO₄³⁻) in the composition. For example, dipotassium phosphate (K₂HPO₄) may be referred to as the potassium ion source or the phosphate ion source because it is capable of providing both the potassium ions and the phosphate ions in the composition.

In the present invention, the composition may exhibit excellent improvement in dentin hypersensitivity while maintaining the stability of the formulation of the composition by comprising 0.10 wt % to 10.00 wt % of a potassium ion source based on the total weight of the composition.

Accordingly, the composition may comprise 0.10 wt % to 10.00 wt %, 0.10 wt % to 9.00 wt %, 0.10 wt % to 8.00 wt %, 0.10 wt % to 7.00 wt %, 0.10 wt % to 6.00 wt %, 0.10 wt % to 5.00 wt %, 0.10 wt % to 4.00 wt %, 0.10 wt % to 3.00 wt %, 0.20 wt % to 10.00 wt %, 0.20 wt % to 9.00 wt %, 0.20 wt % to 8.00 wt %, 0.20 wt % to 7.00 wt %, 0.20 wt % to 6.00 wt %, 0.20 wt % to 5.00 wt %, 0.20 wt % to 4.00 wt %, 0.20 wt % to 3.00 wt %, 0.30 wt % to 10.00 wt %, 0.30 wt % to 9.00 wt %, 0.30 wt % to 8.00 wt %, 0.30 wt % to 7.00 wt %, 0.30 wt % to 6.00 wt %, 0.30 wt % to 5.00 wt %, or 0.30 wt % to 4.00 wt %, and more specifically, 0.10 wt % to 3.00 wt % of the potassium ion source in the composition.

In the present invention, the composition may exhibit an excellent improvement in dentin hypersensitivity while maintaining the stability of the formulation of the composition by comprising 0.10 wt % to 10.00 wt % of a phosphate ion source based on the total weight of the composition.

Accordingly, the composition may comprise 0.10 wt % to 10.00 wt %, 0.10 wt % to 9.00 wt %, 0.10 wt % to 8.00 wt %, 0.10 wt % to 7.00 wt %, 0.10 wt % to 6.00 wt %, 0.10 wt % to 5.00 wt %, 0.10 wt % to 4.00 wt %, 0.10 wt % to 3.00 wt %, 0.20 wt % to 10.00 wt %, 0.20 wt % to 9.00 wt %, 0.20 wt % to 8.00 wt %, 0.20 wt % to 7.00 wt %, 0.20 wt % to 6.00 wt %, 0.20 wt % to 5.00 wt %, 0.20 wt % to 4.00 wt %, 0.20 wt % to 3.00 wt %, 0.30 wt % to 10.00 wt %, 0.30 wt % to 9.00 wt %, 0.30 wt % to 8.00 wt %, 0.30 wt % to 7.00 wt %, 0.30 wt % to 6.00 wt %, 0.30 wt % to 5.00 wt %, or 0.30 wt % to 4.00 wt %, and more specifically, 0.10 wt % to 3.00 wt % of the phosphate ion source in the composition.

In the present invention, the organic zinc compound may be one or more selected from the group consisting of zinc citrate, zinc stearate, zinc gluconate, and zinc carbonate hydroxide, but is not limited thereto.

In the present invention, the organic zinc compound may not include zinc chloride.

In the present invention, the composition may exhibit an excellent whitening effect while exhibiting a tooth enamel strengthening effect by comprising 0.10 wt % to 3.00 wt % of the organic zinc compound, which contributes in filling the gaps between the teeth, based on the total weight of the composition.

Accordingly, the composition may comprise 0.10 wt % to 3.00 wt %, 0.10 wt % to 2.80 wt %, 0.10 wt % to 2.60 wt %, 0.10 wt % to 2.40 wt %, 0.10 wt % to 2.20 wt %, or 0.10 wt % to 2.00 wt %, and more specifically, 0.10 wt % to 1.80 wt % of the organic zinc compound in the composition.

In the present invention, the polyphosphate may be one or more selected from the group consisting of linear polyphosphates, cyclic polyphosphates, and branched polyphosphates, but is not limited thereto.

In the present invention, the polyphosphate may be one or more selected from the group consisting of sodium tripolyphosphate, sodium metaphosphate, and sodium polyphosphate, but is not limited thereto.

In the present invention, the sodium metaphosphate may be sodium hexametaphosphate, but is not limited thereto.

The term “polyphosphate” used herein generally refers to salts of polymeric oxyanions, etc. that share oxygen atoms and are formed from tetrahedral PO₄ structural units linked together. As used herein, the term refers to a component having three or more repeating units of phosphate. The terms “phosphate ions” and “polyphosphate” used herein must be clearly distinguished from each other. For example, sodium hexametaphosphate has the structure of Formula I below.

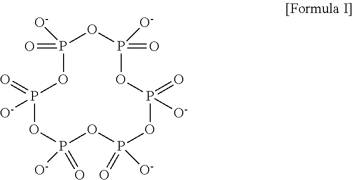

In the present invention, the composition may exhibit low oral irritation and an excellent whitening effect by comprising 1.50 wt % to 10.00 wt % of polyphosphate, which exhibits a tooth whitening effect, based on the total weight of the composition.

Accordingly, the composition may comprise 1.50 wt % to 10.00 wt %, 1.50 wt % to 9.00 wt %, 1.50 wt % to 8.00 wt %, 3.00 wt % to 10.00 wt %, 3.00 wt % to 9.00 wt %, 3.00 wt % to 8.00 wt %, 5.00 wt % to 10.00 wt %, 5.00 wt % to 9.00 wt %, or 5.00 wt % to 8.00 wt %, and more specifically, 7.50 wt % of the polyphosphate in the composition.

In the present invention, the composition may comprise 0.1 wt % to 3.0 wt % of the potassium ion source, 0.1 wt % to 3.0 wt % of the phosphate ion source, 0.1 wt % to 3.0 wt % of the organic zinc compound, and 1.5 wt % to 10.0 wt % of the polyphosphate, but is not limited thereto.

The composition may be applied, without particular limitation, to oral products that may be used for tooth whitening, tooth enamel strengthening, or improving dentin hypersensitivity, which may be, for example, toothpastes, oral sprays, mouthwashes, oral ointments, oral cleansers, chewing gums, candies, oral patches, etc. The oral products may be in the form of a liquid, solid, suspension, gel, film, or patch, but are not limited thereto. When the composition of the present invention is provided in a patch form, the patch may be provided in a form that is orally dissolvable or that can be removed after being attached in the mouth, and the patch may be used in any form without limitation.

A product produced using the composition of the present invention may be prepared using any method commonly used in the art.

In order to apply the composition of the present invention to an oral product, suitable additives, carriers, and the like may be further included in the composition, and a person skilled in the art may appropriately select and combine the additives, carriers, and the like.

The ingredients included in an oral product using the composition of the present invention may include other ingredients commonly used in oral products, for example, abrasive agents, wetting agents, binding agents, foaming agents, sweetening agents, preservatives, active ingredients, flavoring agents, colorants, solvents, whitening agents, solubilizers, or pH adjusters, in addition to the composition comprising a potassium ion, a phosphate ion, an organic zinc compound, and a polyphosphate.

Another aspect of the present invention provides a pharmaceutical composition for preventing or treating dentin hypersensitivity, comprising a composition comprising a potassium ion source, a phosphate ion source, an organic zinc compound, and a polyphosphate.

As used herein, the term “prevention” may refer to any act that inhibits or delays the onset of dentin hypersensitivity by administering the composition.

As used herein, the term “treatment” may refer to any act that improves or beneficially alters dentin hypersensitivity by administering the composition.

The pharmaceutical composition of the present invention may further include a pharmaceutically acceptable carrier.

As used herein, the term “pharmaceutically acceptable” means that it is commonly used in the pharmaceutical field, but it neither causes irritation to a subject upon administration nor inhibits the biological activity and properties of any compounds to be administered.

As used herein, the term “subject” may refer to a mammal, including humans, and may be, for example, humans, monkeys, cows, horses, sheep, pigs, cats, dogs, mice, rats, rabbits, or guinea pigs, but is not limited thereto.

As used herein, the term “administration” refers to the provision of a given substance to a subject by any suitable method, and the pharmaceutical composition of the present invention may be administered through any administration route commonly known in the art as long as it enables the delivery of the pharmaceutical composition to a target tissue.

The present invention may use any carrier commonly used in the art. Non-limiting examples of the carrier may include saline, sterile water, Ringer's solution, buffered saline, an albumin injection solution, lactose, dextrose, sucrose, sorbitol, mannitol, xylitol, erythritol, maltitol, maltodextrin, glycerol, ethanol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, or a combination thereof.

In the present invention, the pharmaceutical composition may include, if necessary, pharmaceutically acceptable additives such as excipients, diluents, antioxidants, buffers, or bacteriostatic agents, and may optionally include fillers, bulking agents, wetting agents, disintegrants, dispersants, surfactants, binding agents, lubricants, etc.

### Advantageous Effects

The present invention relates to an oral composition comprising a potassium ion, a phosphate ion, an organic zinc compound, and a polyphosphate. The present invention exhibits a whitening effect as well as simultaneously exhibiting the effects of strengthening tooth strength and improving dentin hypersensitivity.

### BRIEF DESCRIPTION OF THE DRAWINGS

FIG. 1 is a schematic diagram illustrating the tooth surface remineralization process of the composition according to an embodiment of the present invention.

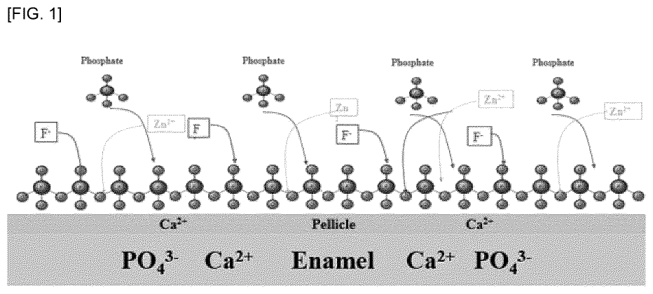

FIG. 2 is a schematic diagram illustrating the process by which potassium ions penetrate the microscopic caps between the teeth to improve dentin hypersensitivity.

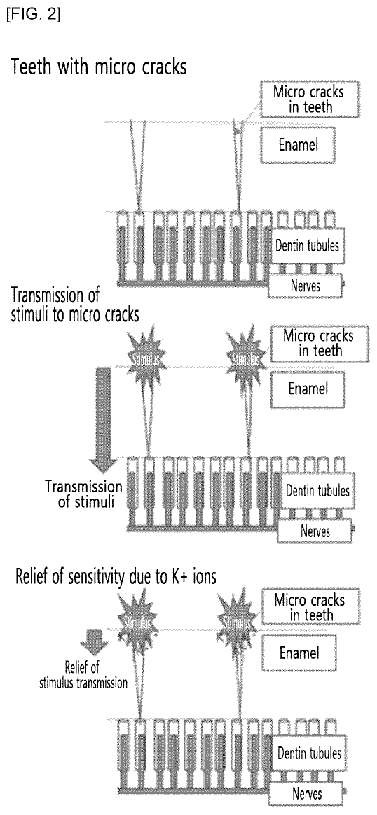

### DETAILED DESCRIPTION OF PREFERRED EMBODIMENTS

Hereinafter, the present disclosure will be described in detail with reference to examples. However, these examples are merely preferable embodiments for illustrating the present disclosure, and therefore, the examples are not intended to limit the scope of right of the present disclosure. Meanwhile, the technical matters not described herein can be sufficiently understood and easily implemented by a person skilled in the art or related art of the present invention.

### Preparation Example: Preparation of Compositions of Examples and Comparative Examples

In order to evaluate the effects of the compositions based on their ingredients and contents thereof, the compositions of Examples 1 to 10 and Comparative Examples 1 to 6 were prepared.

Specifically, glycerin and polyethylene glycol were mixed with carrageenan and xanthan gum. After adding distilled water, small amounts of raw materials were introduced and dissolved. Then, inorganic solids, surfactants, and fragrances were added to prepare the oral compositions. The specific ingredients and their contents are shown in Tables 1 and 2 below.

TABLE 1

| Name of | Example | Example | Example | Example | Example | Example | Example | Example | Example | Example |
|---|---|---|---|---|---|---|---|---|---|---|
| Ingredient | 1 | 2 | 3 | 4 | 5 | 6 | 7 | 8 | 9 | 10 |
| Glycerin | 58.56 | 56.96 | 54.16 | 30.00 | 30.00 | 30.00 | 30.00 | 55.16 | 55.16 | 56.96 |
| Polyethylene | 3.50 | 3.50 | 3.50 | 3.50 | 3.50 | 3.50 | 3.50 | 3.50 | 3.50 | 3.50 |
| glycol | | | | | | | | | | |
| Carrageenan | 0.50 | 0.50 | 0.50 | 0.30 | 0.30 | 0.30 | 0.30 | 0.50 | 0.50 | 0.30 |
| Xanthan gum | 0.30 | 0.30 | 0.30 | 0.50 | 0.50 | 0.50 | 0.50 | 0.30 | 0.30 | 0.50 |
| Sodium fluoride | 0.22 | 0.22 | 0.22 | 0.22 | 0.22 | 0.22 | 0.22 | 0.22 | 0.22 | 0.22 |
| Sodium | 7.50 | 7.50 | 7.50 | 7.50 | 7.50 | 7.50 | — | 7.50 | 7.50 | 7.50 |
| hexametaphosphate | | | | | | | | | | |
| sodium | — | — | — | — | — | — | 7.50 | — | — | — |
| polyphosphate | | | | | | | | | | |
| Sodium | — | — | — | — | — | — | — | — | — | — |
| pyrophosphate | | | | | | | | | | |
| Hydrated silica | 20.00 | 20.00 | 20.00 | 20.00 | 20.00 | 20.00 | 20.00 | 20.00 | 20.00 | 20.00 |
| Monopotassium | 0.10 | 0.40 | 1.00 | 0.10 | 0.40 | 1.00 | 0.40 | — | — | 0.40 |
| phosphate | | | | | | | | | | |
| Tripotassium | 0.20 | 0.80 | 2.00 | 0.20 | 0.80 | 2.00 | 0.80 | — | — | 0.80 |
| phosphate | | | | | | | | | | |
| Potassium nitrate | — | — | — | — | — | — | — | 2.00 | 2.00 | — |
| Trisodium | — | — | — | — | — | — | — | 1.00 | 1.00 | — |
| phosphate | | | | | | | | | | |
| Zinc stearate | 0.10 | 0.80 | 1.80 | 0.10 | 0.80 | 1.80 | 0.80 | 0.80 | — | — |
| Zinc gluconate | — | — | — | — | — | — | — | — | 0.80 | 0.80 |
| Zinc chloride | — | — | — | — | — | — | — | — | — | — |
| Sodium lauryl | 1.50 | 1.50 | 1.50 | 1.50 | 1.50 | 1.50 | 1.50 | 1.50 | 1.50 | 1.50 |
| sulfate | | | | | | | | | | |
| Cocamidopropyl | 1.50 | 1.50 | 1.50 | 1.50 | 1.50 | 1.50 | 1.50 | 1.50 | 1.50 | 1.50 |
| betaine | | | | | | | | | | |
| Tocopheryl | 0.05 | 0.05 | 0.05 | 0.05 | 0.05 | 0.05 | 0.05 | 0.05 | 0.05 | 0.05 |
| acetate | | | | | | | | | | |
| Sodium | 0.22 | 0.22 | 0.22 | 0.22 | 0.22 | 0.22 | 0.22 | 0.22 | 0.22 | 0.22 |
| saccharin | | | | | | | | | | |
| Fragrance | 1.00 | 1.00 | 1.00 | 1.00 | 1.00 | 1.00 | 1.00 | 1.00 | 1.00 | 1.00 |
| Distilled water | 4.75 | 4.75 | 4.75 | 33.31 | 31.71 | 28.91 | 31.71 | 4.75 | 4.75 | 4.75 |

TABLE 2

| | Comparative | Comparative | Comparative | Comparative | Comparative | Comparative |
|---|---|---|---|---|---|---|
| Name of | Example | Example | Example | Example | Example | Example |
| Ingredient | 1 | 2 | 3 | 4 | 5 | 6 |
| Glycerin | 58.56 | 55.96 | 57.16 | 58.96 | 58.16 | 56.96 |
| Polyethylene | 3.50 | 3.50 | 3.50 | 3.50 | 3.50 | 3.50 |
| glycol | | | | | | |
| Carrageenan | 0.50 | 0.50 | 0.50 | 0.50 | 0.50 | 0.50 |
| Xanthan gum | 0.30 | 0.30 | 0.30 | 0.30 | 0.30 | 0.30 |
| Sodium fluoride | 0.22 | 0.22 | 0.22 | 0.22 | 0.22 | 0.22 |
| Sodium | 7.50 | 7.50 | 7.50 | 7.50 | | 7.50 |
| hexametaphosphate | | | | | | |
| Sodium | — | — | — | — | — | — |
| polyphosphate | | | | | | |
| Sodium | — | — | — | — | 7.50 | — |
| pyrophosphate | | | | | | |
| Hydrated silica | 20.00 | 20.00 | 20.00 | 20.00 | 20.00 | 20.00 |
| Monopotassium | — | 1.00 | — | — | — | 0.40 |
| phosphate | | | | | | |
| Tripotassium | — | 2.00 | — | — | — | 0.80 |
| phosphate | | | | | | |
| Potassium nitrate | — | — | — | — | — | — |
| Trisodium | 0.40 | — | — | — | — | — |
| phosphate | | | | | | |
| Zinc stearate | — | — | 1.80 | — | — | — |
| Zinc gluconate | — | — | — | — | 0.80 | — |
| Zinc chloride | — | — | — | — | — | 0.80 |
| Sodium lauryl | 1.50 | 1.50 | 1.50 | 1.50 | 1.50 | 1.50 |
| sulfate | | | | | | |
| Cocamidopropyl | 1.50 | 1.50 | 1.50 | 1.50 | 1.50 | 1.50 |
| betaine | | | | | | |
| Tocopheryl | 0.05 | 0.05 | 0.05 | 0.05 | 0.05 | 0.05 |
| acetate | | | | | | |
| Sodium | 0.22 | 0.22 | 0.22 | 0.22 | 0.22 | 0.22 |
| saccharin | | | | | | |
| Fragrance | 1.00 | 1.00 | 1.00 | 1.00 | 1.00 | 1.00 |
| Distilled water | 4.75 | 4.75 | 4.75 | 4.75 | 4.75 | 4.75 |

### Experimental Example 1: Evaluation of In Vitro Tooth Whitening Effect (Stain Removal Efficacy Evaluation)

The tooth whitening effect of the compositions of the Examples and Comparative Examples was evaluated using hydroxyapatite discs. Hydroxyapatite is a material used in artificial teeth and tissue restoration. The ability of the compositions of the Examples and Comparative Examples to remove contaminants from the hydroxyapatite discs was measured as described below.

Specifically, hydroxyapatite powder was mixed with a polyvinyl alcohol solution and the resultant was compressed with a pressure of 10 tons or more using a compressor. The resultant was then sintered at 1,000° C. to prepare hydroxyapatite discs. The hydroxyapatite discs were stained with coffee, tea, and mucin solutions to prepare specimens, which were then subjected to 5,400 brushings using a brushing machine that applies a 250 g load. Subsequently, stain removal efficacy was evaluated by measuring the color change using a Minolta CR-321 colorimeter. The results are shown in Tables 3 and 4 below.

TABLE 3

| | Example | Example | Example | Example | Example | Example | Example | Example | Example | Example |
|---|---|---|---|---|---|---|---|---|---|---|
| | 1 | 2 | 3 | 4 | 5 | 6 | 7 | 8 | 9 | 10 |
| Stain | 24.10 | 27.78 | 27.26 | 27.22 | 24.13 | 26.20 | 25.87 | 27.12 | 24.46 | 24.24 |
| removing | | | | | | | | | | |
| efficacy | | | | | | | | | | |
| Standard | 4.07 | 4.91 | 6.64 | 5.76 | 6.41 | 6.35 | 5.04 | 6.56 | 4.48 | 5.60 |
| deviation | | | | | | | | | | |

TABLE 4

| | Comparative | Comparative | Comparative | Comparative | Comparative | Comparative |
|---|---|---|---|---|---|---|
| | Example | Example | Example | Example | Example | Example |
| | 1 | 2 | 3 | 4 | 5 | 6 |
| Stain | 16.50 | 18.70 | 12.57 | 25.10 | 15.32 | 13.24 |
| removing | | | | | | |
| efficacy | | | | | | |
| Standard | 6.81 | 6.21 | 5.26 | 4.05 | 5.99 | 6.98 |
| deviation | | | | | | |

As shown in Tables 3 and 4, the stain removal efficacy of the compositions of Examples 1 to 10, which include a potassium ion, a phosphate ion, and an organic zinc compound in addition to a polyphosphate, was measured to range from a minimum of 24.10 (Example 1) to a maximum of 27.78 (Example 2), thereby confirming excellent whitening effects of the compositions.

In particular, it was confirmed that the compositions of Examples 1 to 10 exhibit whitening effects that are similar to, or even superior to, the composition of Comparative Example 4 (25.10), which contains only a polyphosphate.

Moreover, the compositions of Examples 1 to 10 were found to have superior whitening effects compared to the compositions that do not contain a potassium ion (Comparative Examples 1, 3, and 5), compositions that do not contain a phosphate ion (Comparative Examples 3 and 5), and compositions that do not contain an organic zinc compound (Comparative Examples 1, 2, and 6).

Accordingly, it was confirmed that when potassium ions, phosphate ions, organic zinc compounds, and polyphosphates are suitably combined, the whitening effect is improved, whereas when potassium ions, phosphate ions, or organic zinc compounds are used alone, the tooth whitening effect is significantly reduced.

### Experimental Example 2: Evaluation of Tooth Enamel Strengthening

Changes in tooth hardness were evaluated using the compositions prepared in the Preparation Example.

Specifically, for the evaluation method, teeth specimens were prepared by drilling teeth samples into smaller pieces of teeth, followed by mounting and polishing the small teeth with appropriate tools. After etching the specimens with 0.1 M lactic acid, the initial hardness was measured using a Vickers hardness tester. The test specimens were then grouped into eight specimens per group, and the final hardness was measured by performing pH cycling (five cycles) as described below.

pH cycling

| Time | Treatment |
|---|---|
| 09:00~09:05 | Toothpaste* |
| 09:05~10:00 | Saliva** |
| 10:00~10:05 | Toothpaste |
| 10:05~11:00 | Saliva |
| 11:00~15:00 | Acid Challenge*** |
| 15:00~16:00 | Saliva |
| 16:00~16:05 | Toothpaste |
| 16:05~17:00 | Saliva |
| 17:00~17:05 | Toothpaste |
| 17:05~18:00 | Saliva |

*Toothpaste/Saliva = 1/2
**Artificial/Natural = 1/1
***0.1M Lactic Acid Buffer

[symbol]

Microhardness analysis was conducted after caries formation (T1) and after remineralization treatment (T2), and the results are shown in Tables 5 and 6 below.

TABLE 5

| | Example | Example | Example | Example | Example | Example | Example | Example | Example | Example |
|---|---|---|---|---|---|---|---|---|---|---|
| | 1 | 2 | 3 | 4 | 5 | 6 | 7 | 8 | 9 | 10 |
| Change in | 23.29 | 31.95 | 49.00 | 21.55 | 28.49 | 35.51 | 30.86 | 33.39 | 34.64 | 33.11 |
| tooth | | | | | | | | | | |
| hardness | | | | | | | | | | |
| (ΔVHN) | | | | | | | | | | |
| Standard | 5.63 | 5.64 | 3.90 | 5.28 | 3.77 | 5.20 | 3.67 | 4.36 | 3.75 | 3.33 |
| deviation | | | | | | | | | | |

TABLE 6

| | Comparative | Comparative | Comparative | Comparative | Comparative | Comparative |
|---|---|---|---|---|---|---|
| | Example | Example | Example | Example | Example | Example |
| | 1 | 2 | 3 | 4 | 5 | 6 |
| Change in | 12.03 | 21.75 | 25.56 | 9.67 | 9.13 | 18.37 |
| tooth | | | | | | |
| hardness | | | | | | |
| (ΔVHN) | | | | | | |
| Standard | 4.62 | 4.80 | 3.65 | 5.11 | 5.60 | 3.90 |
| deviation | | | | | | |

As shown in Tables 5 and 6 above, it was confirmed that tooth hardness was significantly improved in Examples 1 to 10 compared to that of Comparative Example 4, which does not include a potassium ion, a phosphate, or an organic zinc compound. Further, it was confirmed that using zinc salts stabilized by organic acids demonstrated a higher tooth strengthening effect compared to Comparative Example 6, which includes zinc chloride, where zinc is not stabilized. This suggests that in the case of Comparative Example 6, zinc reacts prematurely in the formulation and thus is unable to contribute to tooth strengthening.

Accordingly, it was confirmed that the composition of the present invention exhibits a tooth hardness strengthening effect as well as having an excellent tooth whitening effect.

### Experimental Example 3: Evaluation of Dentin Hypersensitivity Improvement

An electric pulp test (EPT) was performed on volunteers, and the measurements were collected.

The method of the electric pulp test for measuring dentin hypersensitivity was determined with reference to published literature (KIM, Young-Sung, Journal of Periodontal and Implant Science, Volume 32 Issue 2, 371-378, 2002)), and the result was evaluated to show an improvement in dentin hypersensitivity when the measured values were higher than those from the previous measurements.

In each experiment, a baseline electric pulp test was performed before using the composition, and the test was performed again two weeks after using the composition. The results are shown in Tables 7 and 8 below.

TABLE 7

| | Example 1 | | Example 3 | | Example 6 | | Example 7 | |
|---|---|---|---|---|---|---|---|---|
| | EPT | Standard | EPT | Standard | EPT | Standard | EPT | Standard |
| | index | deviation | index | deviation | index | deviation | index | deviation |
| Base | 10.06 | 5.90 | 13.79 | 5.29 | 11.53 | 4.27 | 13.02 | 4.18 |
| line | | | | | | | | |
| Week 2 | 15.41 | 6.94 | 21.53 | 6.74 | 18.33 | 6.04 | 19.36 | 4.83 |

| | | | | |
|---|---|---|---|---|
| ΔEPT | 5.36 | 7.74 | 6.79 | 6.34 |

TABLE 8

| | Comparative Example 1 | | Comparative Example 4 | |
|---|---|---|---|---|
| | EPT | Standard | EPT | Standard |
| | index | deviation | index | deviation |
| Base line | 12.96 | 6.09 | 13.35 | 6.44 |
| Week 2 | 14.77 | 5.50 | 10.48 | 5.69 |

| | | |
|---|---|---|
| ΔEPT | 1.81 | −2.87 |

As shown in Tables 7 and 8 above, the compositions of Examples 1, 3, 6, and 7, which include potassium ions, phosphate ions, organic zinc compounds, and polyphosphates, showed increased resistance to stimuli compared to those of Comparative Examples 1 and 4, thereby confirming a more significant improvement in dentin hypersensitivity. On the other hand, it was confirmed that the composition lacking a potassium ion, a phosphate ion, or a zinc salt (Comparative Example 4) showed increased sensitiveness to stimuli, resulting in a slight worsening of dentin hypersensitivity.

To summarize the above experimental results, it was confirmed that the composition of the present invention, which includes a potassium ion, a phosphate ion, an organic zinc compound, and a polyphosphate, not only maintains excellent whitening effects but also effectively strengthens tooth enamel and improves dentin hypersensitivity. Therefore, the composition according to the present invention can be usefully applied for tooth whitening, tooth enamel strengthening, and improving dentin hypersensitivity.

As set forth above, a person skilled in the art will be able to understand that the present invention may be embodied in other specific forms without departing from the technical spirit or essential characteristics thereof. In relation thereto, the embodiments described above should be construed as being exemplified and not limiting the present disclosure. The scope of the present invention should be understood to include all changes or modifications derived from the definitions and scopes of the claims and their equivalents, rather than from the detailed description.

1. An oral composition comprising a potassium ion source, a phosphate ion source, an organic zinc compound, and a polyphosphate.

2. The composition of claim 1, wherein the potassium ion source is one or more selected from the group consisting of monopotassium phosphate (KH₂PO₄), dipotassium phosphate (K₂HPO₄), tripotassium phosphate (K₃PO₄), potassium nitrate (KNO₃), potassium chloride (KCl), and potassium oxalate (K₂C₂O₄).

3. The composition of claim 1, wherein the phosphate ion source is one or more selected from the group consisting of monopotassium phosphate (KH₂PO₄), dipotassium phosphate (K₂HPO₄), tripotassium phosphate (K₃PO₄), monosodium phosphate (NaH₂PO₄), disodium phosphate (Na₂HPO₄), and trisodium phosphate (Na₃PO₄).

4. The composition of claim 1, wherein the organic zinc compound is one or more selected from the group consisting of zinc citrate, zinc stearate, zinc gluconate, and zinc carbonate hydroxide.

5. The composition of claim 1, wherein the polyphosphate is one or more selected from the group consisting of linear polyphosphates, cyclic polyphosphates, and branched polyphosphates.

6. The composition of claim 1, wherein the polyphosphate is one or more selected from the group consisting of sodium tripolyphosphate, sodium metaphosphate, and sodium polyphosphate.

7. The composition of claim 6, wherein the sodium metaphosphate is sodium hexametaphosphate.

8. The composition of claim 1, wherein the composition comprises 0.1 wt % to 3.0 wt % of the potassium ion source, 0.1 wt % to 3.0 wt % of the phosphate ion source, 0.1 wt % to 3.0 wt % of the organic zinc compound, and 1.5 wt % to 10.0 wt % of the polyphosphate.

9. The composition of claim 1, wherein the composition is for one or more uses selected from the group consisting of tooth whitening, tooth enamel strengthening, and improving dentin hypersensitivity.

10. An oral product comprising the composition according to claim 1.

11. The oral product of claim 10, wherein the oral product is one or more selected from the group consisting of toothpastes, oral sprays, mouthwashes, oral ointments, oral cleansers, gums, candies, and oral patches.

12. A pharmaceutical composition for preventing or treating dentin hypersensitivity comprising the composition according to claim 1.
